(19) United States
(12) Patent Application Publication
(10) Pub. No.: US 2022/0378865 A1
(43) Pub. Date: Dec. 1, 2022

(54) Formulation for and Method of Using an Herbal Tea for the Treatment of Urinary Tract Infections

(71) Applicants: Daniel Ramley, Chattanooga, TN (US); Grace Ramley, Cottonwood, CA (US)

(72) Inventors: Daniel Ramley, Chattanooga, TN (US); Grace Ramley, Cottonwood, CA (US)

(21) Appl. No.: 17/334,670

(22) Filed: May 28, 2021

(51) Int. Cl. A61K 36/736; A61K 45/06

(52) U.S. Cl. CPC A61K 36/736; A61K 45/06; A61K 2236/331

(57) ABSTRACT

Formulations for and methods using an herbal tea for the treatment and or prevention of UTI and/or kidney infections which may include a combination of ingredients, each with specific medicinal properties, such that the resulting combination is more effective than the individual ingredients alone. Some embodiments of the present invention include an anti-microbial/anti-bacterial ingredient, an anti-inflammatory ingredient, and a relaxation/stress-reducing ingredient, wherein the anti-microbial/anti-bacterial ingredient may be peach leaves.

### CROSS-REFERENCES TO RELATED APPLICATIONS

Not Applicable.

### FIELD OF INVENTION

The present invention relates generally to herbal treatment. More particularly, the present invention is related to a tea for the treatment of urinary tract and kidney infections.

### BACKGROUND OF THE INVENTION

Urinary tract infections (“UTIs”) are relatively common infections that can cause a great deal of discomfort. Typically, urinary tract infections are treated with medications and antibiotics. However, there are patients who prefer to avoid such treatment. Firstly, it has been shown that over reliance and improper use of antibiotics has led to antibiotic resistant strains of bacteria that pose a greater health risk to the public. Additionally, some patients simply prefer a natural, wholistic alternative to industrially produced pharmaceutical treatments.

Therefore, there exists a need for an improved herbal treatment for urinary tract, kidney, and other internal infections.

### SUMMARY OF THE INVENTION

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter. The present invention is defined by the claims.

The embodiments of the present invention are directed towards formulations for and methods using an herbal tea for the treatment and or prevention of UTI and/or kidney infections. The present invention provides a 100% all natural treatment for UTI and/or kidney infections that obviates the need for manufactured medications or antibiotics.

In some embodiments, of the present invention may include a combination of ingredients, each with specific medicinal properties, such that the resulting combination is more effective than the individual ingredients alone. Some embodiments of the present invention include an antimicrobial/antibacterial ingredient, an anti-inflammatory ingredient, and a relaxation/stress-reducing ingredient. In some embodiments the antimicrobial/antibacterial ingredient may be peach leaves.

### BRIEF DESCRIPTION OF THE DRAWINGS

The present invention is described in detail below with reference to the attached drawing FIGURES, wherein:

FIG. 1 illustrates a flowchart corresponding to a method performed according to an embodiment of the present invention.

### DETAILED DESCRIPTION OF THE INVENTION

Some embodiments of the present invention are described with specificity herein to meet statutory requirements. However, the scope of the invention is not intended to be defined by the description itself. The claimed subject matter may be embodied as to include different features, elements, components, steps, or combinations of steps, similar to those described herein, and in combination with other existing or future technologies. Moreover, although the term “step” might be used to connote different elements of the methods employed, this term should not be interpreted as implying any particular order among or between various steps herein disclosed unless and except the order of individual steps is explicitly described or required.

Peach leaves have antimicrobial and antibacterial properties which help get rid of UTI and/or kidney infection. While effective on its own, inclusion of ingredients with complementary medicinal properties greatly improves the effectiveness of the tea. For example, the effectiveness of the tea may be aided by the anti-inflammatory properties in the rose buds, rose hips and chamomile. These ingredients help reduce inflammation caused by UTI or kidney infection. In addition, chamomile also aids in relaxation which enables the body to have more adequate rest so it can heal faster. One having skill in the art will recognize that stress inhibits the body's natural immune response, thus ingredients that reduce stress improve recovery from infections.

In some embodiments of the present invention, the combination of ingredients may include at least one antimicrobial/antibacterial ingredient, at least one anti-inflammatory ingredient, and at least one relaxation/stress-relieving ingredient. The combined effect of each of these medicinal herbal ingredients may serve to increase the effectiveness of the resulting teas for the treatment of UTI and/or kidney infections. Examples of antimicrobial/antibacterial ingredients include, but are not limited to peach leaves, thyme, wormwood, mint, pomegranate blossom, black tea, oregano, cinnamon, sage, and echinacea. Examples of anti-inflammatory ingredients include, but are not limited to rooibos, honeybush, green tea, ginger, rose hips, rose buds, and turmeric. Examples of relaxation/stress-relieving ingredient include, but are not limited to chamomile, lavender, passionflower, valerian root, and lemon balm.

Ingredients may be prepared through a process of washing and drying each ingredient. In some embodiments, each ingredient is quadruple washed and drip dried. In such embodiments, each ingredient may then be placed in a dehydrator at 125° F. for 12 hours to dehydrate the ingredients while maintaining the nutrients therein.

In some embodiments, a preparation of the tea may include 50% by mass of at least one antimicrobial/antibacterial ingredient, 40% by mass of at least one anti-inflammatory ingredient, and 10% by mass of at least one relaxation/stress-relieving ingredient. For example, one embodiment of the present invention may include 50% by mass of peach leaves as the antimicrobial/antibacterial ingredient, 30% by mass of rose hips and 10% by mass of rose buds as the anti-inflammatory ingredients, and 10% by mass of chamomile as the relaxation/stress-relieving ingredient.

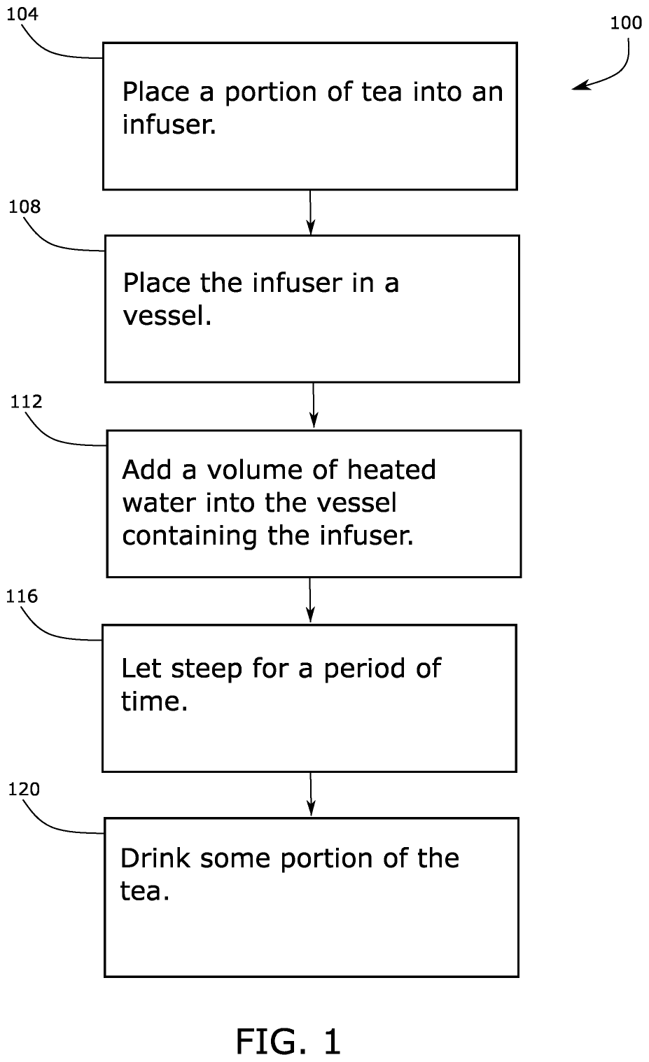

FIG. 1 illustrates a flowchart 100 corresponding to a method performed according to an embodiment of the present invention. At step 104, a particular portion of dried tea ingredients are place into an infuser. An infuser may include, but is not limited to a ball-style mesh infuser, a basket style mesh infuser, the French-press style infuser, a tea bag, a cheesecloth sachet bag, or the like. The particular portion of dried tea ingredients may depend upon the volume of water to be added. For example, in some embodiments, the particular portion of dried tea ingredients may be ½ tablespoon of tea per 8-12 fluid ounces of water.

At step 108, the infuser is placed into a vessel. Suitable vessels include, but are not limited to a teacup, a coffee cup, a tea bowl, a teapot, or the like. At step 112, a volume of heated water is added to the vessel containing the infuser. In some embodiments, the volume of heated water may be heated to 212° F.

At step 116, the herbal tea ingredients is left to steep in the water for a period of time to produce a tea. In some embodiments, the tea may be left to steep for 4 minutes. At step 120, a user may drink some portion of the tea per some interval of time. In some embodiments, the user may drink 1 cup each hour of the tea until symptoms, such as pain, dissipate. In some embodiments, the user may drink at least 8 cups over 8 hours to receive full benefit of the tea.

In some embodiments, the present invention may be used preventively when the user believes that extra urinary tract support may be necessary. For examples, a user may believe extra support is necessary after exercise, after sexual intercourse, or other activity associated with a heightened risk of UTI and/or kidney infection.

Embodiments of the present invention have been described, as required by statute, to be illustrative, but should not be interpreted to be restrictive. One having skill in the art will recognize that many different arrangements of the various components depicted are possible without departing from the scope of the claims below, as well as arrangements including components not explicitly shown.

One having skill in the art will understand that certain combinations and/or sub-combinations of elements and features are of utility and may be employed without reference to other combinations and/or sub-combinations and are contemplated within the scope of the claims. Not all steps listed in the various FIGURES need be carried out in the specific order described.

What is claimed is:

1. An herbal tea for preventing or treating infection comprising:
at least one antimicrobial/antibacterial ingredient;
at least one anti-inflammatory ingredient; and
at least one relaxation/stress-receiving ingredient.

2. The herbal tea of independent claim 1, wherein:
the at least one antimicrobial/antibacterial ingredient is at least one of peach leaves, thyme, wormwood, mint, pomegranate blossom, black tea, oregano, cinnamon, sage, and echinacea.

3. The herbal tea of independent claim 1, wherein:
the at least one anti-inflammatory ingredient is at least one of rooibos, honeybush, green tea, ginger, rose hips, rose buds, and turmeric.

4. The herbal tea of independent claim 1, wherein:
the at least one relaxation/stress-relieving ingredient is at least one of chamomile, lavender, passionflower, valerian root, and lemon balm.

5. The herbal tea of independent claim 1, wherein:
the at least one antimicrobial/antibacterial ingredient is approximately 50% by mass of the herbal tea.

6. The herbal tea of independent claim 1, wherein:
the at least one anti-inflammatory ingredient is approximately 40% by mass of the herbal tea.

7. The herbal tea of independent claim 1, wherein:
the at least one relaxation/stress-relieving ingredient is approximately 10% by mass of the herbal tea.

8. A method of treating an infection comprising:
place a portion of an herbal tea into an infuser, the herbal tea comprising at least one antimicrobial/antibacterial ingredient, at least one anti-inflammatory ingredient, and at least one relaxation/stress-receiving ingredient;
placing the infuser in a vessel;
adding a volume of heated water;
leaving the herbal tea to steep in the heated water for a period of time to produce a tea; and
having a user drink a portion of the tea per an interval of time.

9. The method of treating an infection of independent claim 8, wherein:
at least one antimicrobial/antibacterial ingredient comprises peach leaves.

10. The method of treating an infection of independent claim 8, wherein:
the portion of herbal tea is ½ tablespoon, and
wherein the volume of heated water is between 8 fluid ounces and 12 fluid ounces.

11. The method of treating an infection of independent claim 8, wherein:
the heated water is heated to a temperature of 212° F.

12. The method of treating an infection of independent claim 8, wherein:
the portion of the tea is a cup, and
wherein the interval of time is one hour.
